(19) United States
(12) Patent Application Publication
(10) Pub. No.: US 2005/0240149 A1
(43) Pub. Date: Oct. 27, 2005

(54) Self-destructive syringe

(76) Inventor: Chun Hui Lu, Jhongli City (TW)

Correspondence Address: TROXELL LAW OFFICE PLLC, SUITE 1404, 5205 LEESBURG PIKE, FALLS CHURCH, VA, 22041, US

(21) Appl. No.: 10/829,966

(22) Filed: Apr. 23, 2004

(51) Int. Cl. A61M 5/00

(52) U.S. Cl. 604/110; 604/218

(57) ABSTRACT

A self-destructive syringe including a barrel, a plunger in the barrel, and a needle head adapted with a needle, a plunger holder at the front end of the plunger comprised of a primary anda secondary tapers each provided with a ring plug, a fixation ring holder and a locking member; a hollow needle holder formed at the front end of the barrel to accommodate the needle head; a limiting part in the needle holder; upon completing the injection, both of the ring plug, the secondary taper, the fixation ring holder and the locking member being left in the barrel, and the plunger and the primary taper being pulled out of barrel to prevent reuse of the syringe.

### BACKGROUND OF THE INVENTION

(a) Field of the Invention

The present invention is related to a self-destructive syringe, and more particularly, to one that when disposed prevents any attempted recycle for use.

(b) Description of the Prior Art

The conventional syringe permits repeated use after sterilization; but any improper sterilization leads to possible infection. To prevent such infection, a disposal syringe has been developed. However, the structure of the disposed syringe remains intact and the risk of its recycled use does present.

### SUMMARY OF THE INVENTION

The primary purpose of the present invention is to provide a self-destructive syringe that prevents any attempts of repeated use of the syringe when recycled. To achieve the purpose, the present invention includes a barrel, a plunger in the barrel, and a needle. A plunger holder provided at the front end of the plunger is comprised of a primary taper, a secondary tape, a fixation ring holder and a locking member in sequence. A ring plug is each provided on the primary and the secondary tapers. A hollow needle holder is formed at the front end to accommodate a needle head, and a limiting part is formed in the needle holder. Accordingly, upon completing the injection, the plunger is pushed all way down to the end for the locking member to enter into the needle holder and to be interlocked with the limiting part. The plunger is then pulled back to separate both of the primary and the secondary tapers from each other to leave the ring plug, the secondary tape and the fixation ring holder stuck in the needle holder by means of the locking member so that the ring plug, the secondary tape, the fixation ring holder and the locking member are all staying in the barrel while the plunger and the primary taper are pulled out of the barrel to prevent the syringe from repeated use.

### BRIEF DESCRIPTION OF THE DRAWINGS

FIG. 1 is a perspective view of a preferred embodiment of the present invention.

FIG. 2 is an exploded view of the preferred embodiment of the present invention.

FIG. 3 is a schematic view of the preferred embodiment of the present invention.

FIG. 4 is a schematic view showing a plunger and a needle holder of the preferred embodiment.

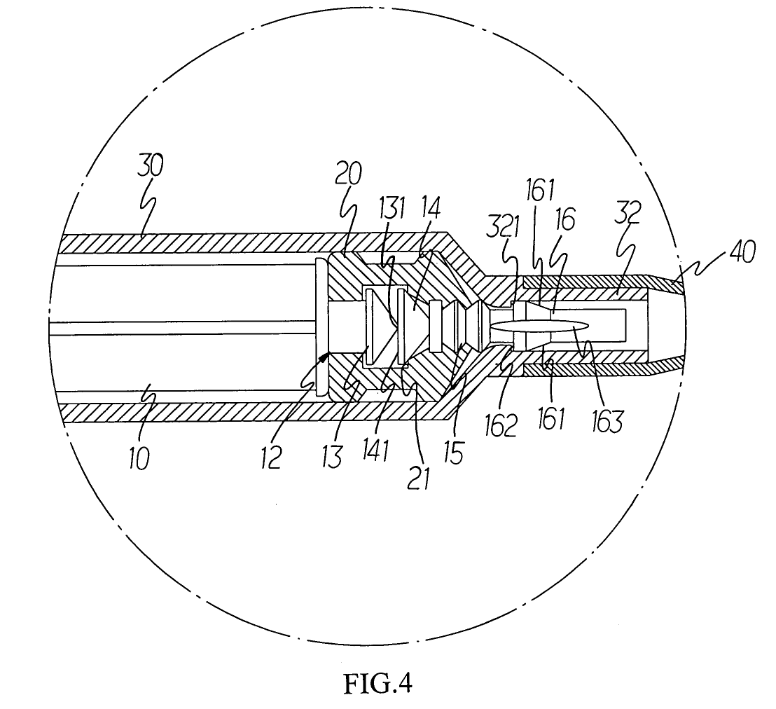

FIG. 5 is a schematic view showing the plunger is pulled back.

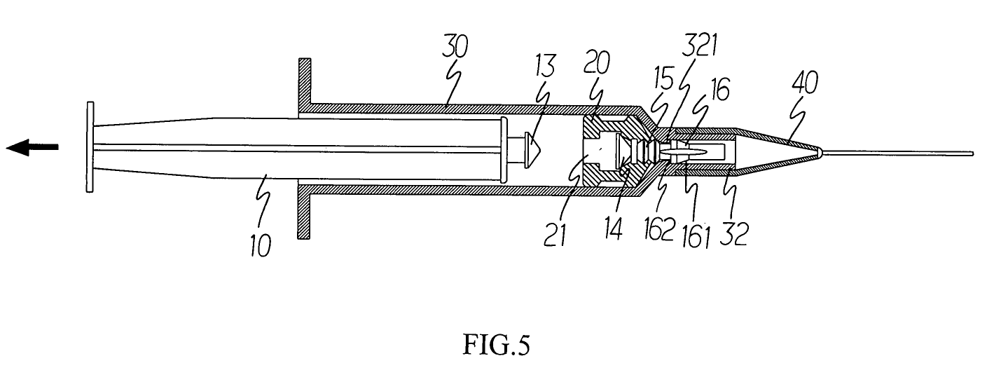

### DETAILED DESCRIPTION OF THE PREFERRED EMBODIMENTS

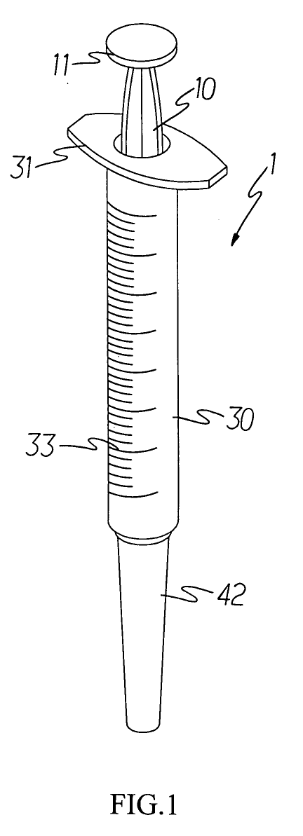

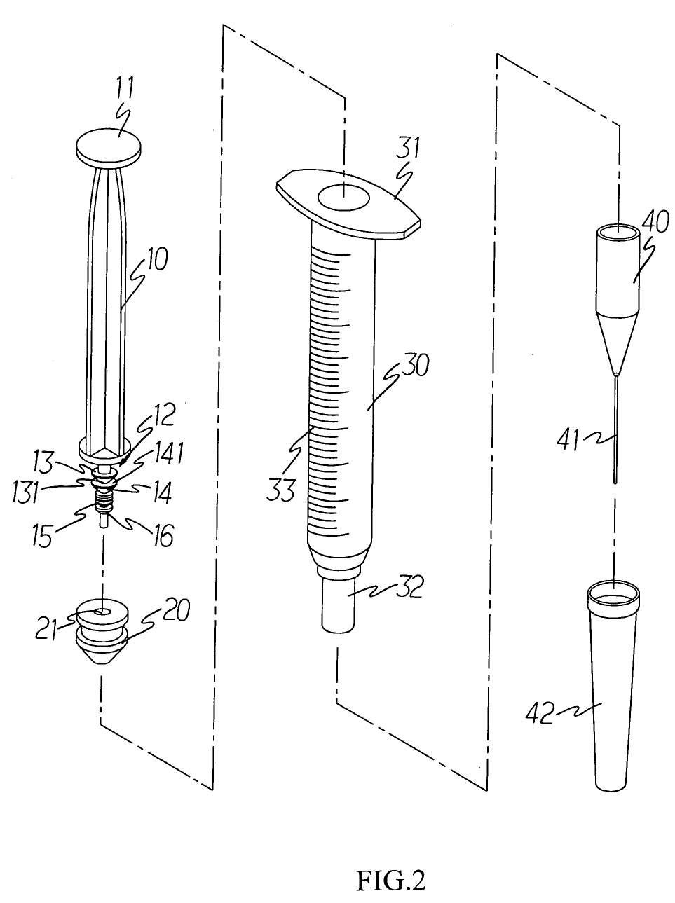

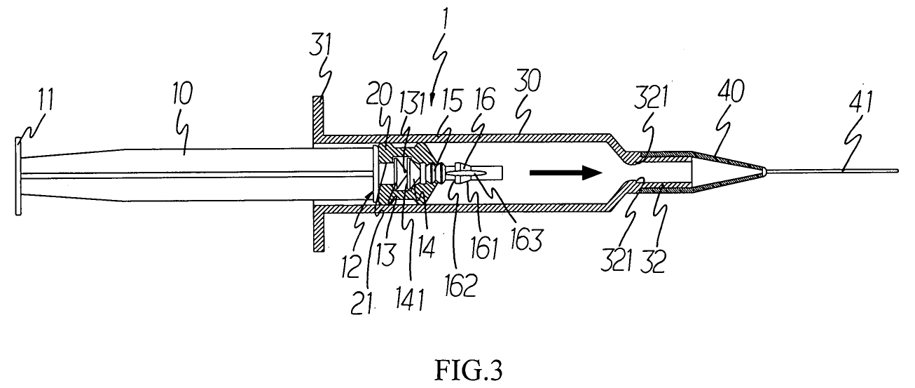

Referring to FIGS. 1-3, a preferred embodiment of the present invention is comprised of a syringe 1 includes a plunger 10, a barrel 30 and a needle holder 40. Wherein, a flange 11 is provided at the rear end of the plunger 10, and a plunger holder 12 is disposed at the front end of the plunger 10. The plunger holder 12 is provided in sequence a primary taper 13, a secondary taper 14, a fixation ring holder 15 and a locking member 16. The primary taper 13 has its tip 131 to contact a base 141 of the secondary taper 14. A ring plug 20 is provided to the primary taper 13, the secondary taper 14, and the fixation ring holder 15 so that the primary taper 13, the secondary taper 14 and the fixation ring holder 15 are inserted through a hole 21 provided on the ring plug 20. The locking member 16 is exposed out of the front end of the ring plug 20. A guide 161 is formed at where appropriately on the locking member 16. One side of the guide 161 facing the fixation ring holder 15 is externally enlarged; a locking side 162 is formed at the bottom of the guide 161, and an opening 163 is provided at where appropriately on the locking member 16.

The barrel 30 has at its rear end provided with a flange 31 and at its front end formed with a hollow needle holder 32 for the insertion of a needle head 40. A limiting part 321 is formed inside the needle holder 32 and a scale 33 is provided on the body of the barrel 30.

The needle head 40 has its one end fixed to a needle 41 and is received in the needle holder 32 while a cap 42 is inserted onto the needle 41.

Accordingly, upon completing the injection of the liquid in the barrel 30, the plunger 10 is further pushed all way down the barrel 30. As guided by the guide 161 of the locking member 16 provided at the front end of the plunger, and the resilient force from the opening 163, the locking member 16 is pushed into the needle holder 32 for the locking side 162 and the limiting part 321 to interlock to each other. The plunger 10 is then pulled back and the resultant pull causes both of the primary and the secondary tapers 13, 14 to separate from each other. The ring plug 20, the secondary taper 14 therein and the fixation ring holder 15 are stuck in the needle holder 32 by means of the locking member 16 while the ring plug 20, the secondary taper 14, the fixation ring holder 15 and the locking member 16 all are left in the barrel 30, and the plunger 10 and the primary taper 13 is pulled out of the barrel 30 to prevent any attempted repeated use of the syringe.

The present invention by providing an improved structure of a self-destructive syringe prevents any attempted reuse of a disposed syringe. Therefore, this application for a utility patent is duly filed accordingly. However, it is to be noted that the preferred embodiment and accompanying drawings disclosed in the specification do not in any way limit the teaching of the present invention. Therefore, any structure, installation, characteristics that are identical with or similar to those taught by the present invention shall be deemed as falling within the scope of the purpose of and claims made by the present invention.

1. An improved structure of a self-destructive syringe includes a plunger, having at its rear end disposed with a flange and its front end, a plunger fixation holder; the plunger holder being provided in sequence a primary taper, a secondary taper, a fixation ring holder and a locking member with the primary taper having its tip to contact a base of the secondary taper; a ring plug being provided to the primary taper, the secondary taper, and the fixation ring holder so that the primary taper, the secondary taper and the fixation ring holder being inserted through a hole provided on the ring plug; the locking member being exposed out of the front end of the ring plug; a barrel having at its rear end provided with a flange and at its front end formed with a hollow needle holder for the insertion of a needle head; a limiting part being formed inside the needle holder; and a needle head having its one end fixed to a needle and received in the needle holder; upon completing the injection of the liquid in the barrel, the plunger being further pushed all way down the barrel; the locking member being pushed into the needle holder to be interlocked with the limiting part; the plunger being pulled back to separate both of the primary and the secondary tapers from each other; the ring plug, the secondary taper therein and the fixation ring holder being stuck in the needle holder by means of the locking member; the ring plug, the secondary taper, the fixation ring holder and the locking member all being left in the barrel, and the plunger and the primary taper being pulled out of the barrel to prevent any attempted reuse of the syringe.

2. An improved structure of a self-destructive syringe as claimed in claim 1, wherein, a guide is formed at where appropriately on the locking member; one side of the guide facing the fixation ring holder being externally enlarged; and a locking side being inwardly formed at the bottom of the guide.

3. An improved structure of a self-destructive syringe as claimed in claim 1, wherein, an opening is provided at where appropriately on the locking member.

4. An improved structure of a self-destructive syringe as claimed in claim 1, wherein, a scale is provided on the body of the barrel.

5. An improved structure of a self-destructive syringe as claimed in claim 1, wherein, a cap is provided to cover up the needle of the needle head.
